(19) United States
(12) Patent Application Publication
(10) Pub. No.: US 2026/0042685 A1
(43) Pub. Date: Feb. 12, 2026

(54) LARGE-SCALE UV-LED FLUID TREATMENT DEVICE

(71) Applicant: AquiSense Inc., Erlanger, KY (US)

(72) Inventors: Jennifer Godwin Pagan, Charlotte, NC (US); Steven Franklin Pugh, Charlotte, NC (US); John Krause, Charlotte, NC (US); Richard Mark Simons, London (GB)

(21) Appl. No.: 19/139,620

(22) PCT Filed: Dec. 18, 2023
(86) PCT No.: PCT/US2023/084613

§ 371 Date: Jun. 16, 2025

Related U.S. Application Data
(60) Provisional application No. 63/433,330, filed on Dec. 16, 2022.

(51) Int. Cl. C02F 1/32; A61L 2/10

(52) U.S. Cl. CPC C02F 1/325; A61L 2/10; A61L 2202/11; A61L 2202/122; A61L 2202/14; C02F 2201/3222; C02F 2201/3228; C02F 2201/326; C02F 2303/04

(57) ABSTRACT

The use of arrays of light emitting diodes (LEDs) emitting in the ultraviolet spectrum (UV-LEDs) for disinfection of large-scale water flow. An irradiation chamber can utilize the high power density of UV-LEDs arranged into a planar array. irradiating fluid flowing through a cylindrical irradiation chamber. formed of a transmissive material. The transmissive irradiation chamber may be enclosed within a secondary optical chamber of a reflective material. or coated or covered with a reflective material. The cylindrical irradiation chamber allows for the deployment of vortex generators to introduce a swirling laminar flow region which causes the flow to spiral along the length of the irradiation chamber to equalize UV exposure between hot spots and dark regions or irradiation.

### CROSS-REFERENCE TO RELATED APPLICATIONS

The present application is a national stage entry of International Application No. PCT/US2023/084613 filed Dec. 18, 2023, which claims the benefit of U.S. Provisional Application No. 63/433,330 filed Dec. 16, 2022, the disclosures of which are hereby incorporated by reference in their entireties.

### FIELD OF THE INVENTION

The present invention relates in general to the use of ultraviolet (UV) light for the disinfection and treatment of fluids, and in particular to the use of arrays of light emitting diodes (LEDs) emitting in the ultraviolet spectrum (UV-LEDs) for disinfection of large-scale water flow.

### BACKGROUND OF THE INVENTION

Liquid compositions can be illuminated with UV light in an in-line format, for example, within piping, tubing, channel, or similar apparatus for transporting a liquid stream from one process, tank, or container to another. Such internal UV-light apparatuses are well-known in the food and water treatment industries. Several patents and patent publications that describe such fluid radiation treatment systems include U.S. Pat. Nos. 4,317,041, 4,482,809, 4,872,980, 5,006,244, 5,418,370, 5,539,210, 5,846,437, 5,866,910, 5,994,705, 6,015,229, 6,916,452, 7,166,850, 7,390,225, 7,695,675, 7,985,956, 8,167,654, and 8,766,211, the disclosures of which are incorporated by reference in their entireties. Turbidity is a measurement of suspended matter in the composition, which can affect microbial kill by shielding pathogens from being contacted by the UV light. Consequently, as a composition becomes more turbid, an effectively larger dose is required to attain a desired microbial kill relative to a composition with a lower turbidity.

Apparatuses and systems for disinfection of liquid using UV light fall into several categories based on the flow rate of the system. Point of Use systems for instance are usually small systems installed near the exit point of the fluid (e.g. a water disinfection system on a single faucet) These systems have low flow rates of fluid passing through the system and typically do not require higher amounts of UV radiation to disinfect the fluid. Point-of-entry systems have higher flow rates and serve multiple points of distribution (e.g. multiple faucets and water to whole home appliances). Industrial systems are typically used in commercial applications, such as water used for a factory process. Municipal systems are the largest systems, and are used prior to distributing drinking water to a community. The table below is typical of drinking water or cooling water system flow rates and required radiometric UV power to provide disinfection to the water.

TABLE 1
Typical Water Disinfection Systems

| | | | Typical Radiometric |
|---|---|---|---|
| Category of System | Typical Flow Rates | | Ultraviolet Power (Watts) |
| Point of Use | Less than 3 GPM | | <1 |
| Point of Entry | 3-12 | GPM | 1-10 |
| Industrial | 50-500 | GPM | 20-400 |
| Municipal | >500 | GPM | >100 |

There exist many other fluid disinfection applications where the ultraviolet transmittance (UVT) of the fluid is lower than 80%, and those applications require more radiometric UV power to achieve disinfection of the fluid. These fluids could be medicines, fruit juices, mining waters, or waste waters. Similarly, there exist photochemical applications where the same principals of scale apply. Disinfection wavelengths can vary, but are generally in the UVC range of 200 nm-300 nm.

Historically, most UV sources were created using mercury-based vapor lamp technology. But in the past 20 years planar ultraviolet sources have been developed that are semiconductor based. The most commercialized example is a UV-LED, i.e., a light emitting diode (LED) emitting in the ultraviolet spectrum; however, UV laser diodes, micro plasma lamps, and electroluminescent devices are alternate examples. The power level and efficiency of these planar sources are still lower than that of mercury-based sources, which have typical electrical conversion efficiencies of 15% and 30% for medium pressure and low-pressure lamps, respectively.

The commercial use of UVC LEDs for example, are progressing rapidly, but current commercial devices have less than 10% electrical conversion efficiency, and they typically must be cooled adequately or else the lifetime, output power, and reliability of the LED will drop significantly. In contrast, mercury vapor lamps operate well at higher temperatures and have trouble with reliability in very cold conditions. In addition, mercury vapor lamps emit heat in the same direction that they emit photons. This is not the case for semiconductor-based UV sources which emit heat from the electrical contacts of the device which are typically connected through the back side of surface mount package. This creates UV sources which emit heat from the side of electrical attachment and emit UV radiation from a different emitting surface, typically the topside of a surface mount package.

UVC LED sources currently have a higher radiometric power density as compared to mercury vapor lamps. The power density can be calculated by the radiometric output power divided by the surface area of the emitting surface of the source. In the case of the UVC LED that would be the topside surface area of the package, while the mercury lamp would use the surface area of the quartz in the calculation.

The higher power density and cooling requirements offer both a design opportunity and a problem to solve when using semiconductor-based UV sources in industrial or municipal sized UV systems. On one hand there are kilowatts of heat to remove from the UV sources, but you can position the UV sources in a smaller footprint and use features such as flow conditioners (vanes, baffles, constrictions, flares), reactor length, and reflectors to increase the efficiency of the delivered UV fluence to the fluid.

The use of arrays of planar sources which exist in a localized position, and whose total surface area covers substantially less than all of the external surface area of the irradiation chamber, create a problem for monitoring the UV flux inside the reactor. For municipal and industrial systems which are critical to protect health or process viability, it is often required to monitor the level of UV flux inside the reactor, so that if the UV is too low an alarm can be relayed to a control system to stop the process to replace the UV source(s). This is further complicated when the UV transmittance of the fluid inside the reactor is low (i.e. the fluid has a high turbidity) and there is more than one planar array located at a different tangent around the reactor. The location of the sensor depends on the tangent of the array, the length of the array, and the reflector positions.

Ultraviolet reactors operate by a photochemical effect, and as such follow the first law of photochemistry Grotthuss-Draper law: photons must be absorbed by a target species for a reaction to occur. Therefore, a basic optimization of reactive efficiency requires maximal absorbance of UV radiation delivered to the fluid to be treated. In the most simplistic case, the rate of reaction (k) is proportional to the power delivered (P_{b}, W) per volume (V, m³): k∝P_{b}/V.

In the case of a UV disinfection reactor in particular, though this method is more generally applied through fluid exposure, the degree of reaction is described via the fluence rate H′ₑ, W m⁻²), requiring the inclusion of the attenuation coefficient base e (αₙ, m⁻¹): H′ₑ=P_{b}/(αₙ V).

Integrating the above over a given period of time provides the fluence (Hₑ, J m⁻²) as a function of flow rate through the reactor (Q, m³ s−1) or as the radiant energy absorbed (W_{b}, J). Disinfection reactions commonly produce log-linear inactivation effect per unit fluence delivered, and so the rate of reaction is often not proportional to fluence, though a positive correlation always exists: Hₑ=P_{b}/(αₙ Q)∥Hₑ=W_{b}/(αₙ V)

The above equations apply either where an excess of “reagent” exists, such that the rate of reaction is constant over the period of time observed, and where the fluence rate is uniform throughout the volume to be treated. These conditions are generally satisfied for short time periods and small evaluation volumes. Where larger volumes are considered, elements of physical reactor design such as divergent radiation sources and photochemical characteristics of the fluid to be treated (attenuation in accordance with the Beer-Lambert law), will result in non-uniformities of fluence rate (He) across the volume of interest. Fluence, or radiant exposure, is the radiant energy received by a surface per unit area, or equivalently the irradiance of a surface, integrated over time of irradiation.

Accordingly, this distribution of fluence rate through a static fluid will result in a distribution of reaction rate, and so a distribution of “product” formation through a volume of the fluid. For a first order reaction, this may still result in an optimized reaction if the end product is mixed, since the volumetric average concentration of “product” is proportional to the volumetric radiant dose (J m⁻³). However, most applications exhibit either a saturation of first order kinetics (e.g. via complete conversion in over-exposed regions), inherently higher-order reaction kinetics (e.g. log-linear inactivation response of most microbes), deleterious or reverse processes (e.g. degradation of ‘product’ in a dynamic equilibrium), or others. In these cases, the distribution and magnitude of fluence delivered through the fluid can significantly affect reaction efficiency and therefore directly impact operating costs.

Considering that management of operational conditions (fluid absorbance, temperature, turbidity, chemical/photochemical activity, etc.) is distinct to the reactor design, there are fundamentally four means of enhancing photochemical reactor efficiency. These means may be applied independently or in concert, and to varying degrees in any given application:
(iv) Selection of a more preferential spectral characteristic of the radiation source to maximize the rate of reaction via a higher quantum yield (Φ(λ), −);
(v) Maximization of the optical coupling between the radiation source and the target material, P_{b}→P₀;
(iii) Maximization of the uniformity of fluence rate distribution through the volume of fluid to be irradiated; and
(vi) Introduction of volumetric mixing to optimally move each volume element of fluid through each volume element of space with a non-zero fluence rate.

In consideration of a preferential spectral characteristic of the radiation source, numerous radiation sources exist, and may be selected, based on their spectral characteristics alone. Additionally, other non-limiting characteristics such as material composition, ruggedness, price, electronic efficiency, power density, and form factor, may be considered. In particular, light emitting diodes (LEDs) emitting in the ultraviolet spectrum (UV-LEDs) may have preferential characteristics for many photochemical applications; notably, their spectral tunability (commercial UV-LEDs have been produced with peak wavelengths from ˜230-400 nm), power density (radiant flux per unit area of device footprint), and modularity (ability to be assembled in series-parallel arrays) may lend themselves to photochemical reactors. The beneficial characteristics of LED sources are well known.

In consideration of the maximization of the optical coupling of the reactor, this may be described either in positive terms (maximizing Pb by efficiently transmitting radiant flux from the source to the target fluid) or in negative terms (minimizing power losses via absorption by elements of the reactor other than the target fluid). The first sense may be referred to as optical in-coupling, where the environment around the source is engineered to preferentially direct radiation towards the reaction chamber containing the fluid (material) to be irradiated. This may be considered by highly technical means, e.g. parabolic reflectors, “beam shaping”, collimation, refractive index matching, etc., or by more simplistic approaches such as reduction of the displacement of the source from the irradiation target to counteract divergence effects. In each of these approaches the intent is direct radiation into the reaction chamber containing the fluid to be irradiated. Inefficient optical in-coupling may be considered as a loss mechanism. In the second sense, the objective is to retain radiant power within the target fluid once optical in-coupling has been achieved. The speed of light is constant for a given medium, and so radiation is always in motion; the only means for retaining radiant power within a medium is therefore for it to be absorbed by that media (recall coincidence with the first law of photochemistry for consequences on photoactivity). However, the absorbance (m⁻¹) is typically fixed for a given medium in a photochemical reactor, meaning that only a given fraction of the radiant power may be absorbed per unit length of propagation.

The reactor designer may consider several options to maximize this path length, and to maximize absorption (radiant power retention), such as: increasing reaction chamber dimensions; positioning of the radiation source relative to the reaction chamber or optical aperture; size and location of optical, hydraulic, or other apertures; material selection for the chamber materials (considering reflective, refractive, photochemical, or photoactive properties). Refractive index boundaries may be applied to utilize phenomena such as total internal reflection within the fluid, equally, highly reflective materials may be applied as part of the reaction chamber (or even beyond transmissive interfaces) to recover radiation back into the target fluid that might otherwise have been lost from the system. Note, that any radiation absorbed by elements of the reactor/system which are not the target fluid or otherwise photoactive, photocatalytic, etc., constitute a loss in optical coupling efficiency.

In consideration of the maximization of fluence rate uniformity, there are several means for manipulating the fluence rate distribution through the reaction chamber, including but not limited to: optimization of the shape and size of the reaction chamber; optimizing the position of the radiation source relative to the reaction chamber; employing beam shaping or optical conditioning of the radiation emitted by the source prior to entrance into the reaction chamber; employment of reflective or diffusing elements to spread radiation through the reaction chamber; employing an integrating cavity approach to produce multiple passes of the radiation along different paths through the fluid within the reaction chamber.

In consideration of volumetric mixing of the process fluid to be irradiated, the fundamental goal is to counteract a distribution in fluence rate through the reaction chamber by flowing the fluid in such a manner than each streamline through the chamber would integrate an equal fluence, and so a uniform fluence is delivered across the entire flow. Some non-uniformity in fluence delivery shall always exist. The trivial solution is for ideal mixing of the fluid through the entire reaction chamber (H′ₑ>0) such that each volume element of the fluid is exposed for an equal period of time to each region of the fluence rate field. Ideal mixing is largely a theoretical upper limit and rarely practically achieved for reaction chambers. More complex designs may consider structured flow from the reactor inlet to the outlet, such as: linear laminar flow through a reaction chamber of regular cross-section; swirling laminar flow through a reactor with curved wall sections; turbulent flow about “sharp” boundaries within the reaction chamber; turbulent flow for the disruption and mixing of boundary layers with central flow regions; amongst other flow concepts. These states of fluid flow may be achieved by manipulating the reaction chamber shape and size, introduction of flow conditioning elements (e.g. baffles, constrictions, flares, vortex generators, static mixers, etc.), manipulating the flow rate through a reaction chamber using parallelized treatment streams, or other means.

Large scale UV fluid treatment, such as municipal UV drinking water disinfection, requires a very high flux of UV light. Inefficiencies in UV light generation result in a large amount of heat generation that must be removed to preserve device function. Municipal applications of UV treatment devices often require that the treatment device is located outdoors or in an otherwise non-climate-controlled environment. For example, a device may be installed outside in a desert. Without control over the exterior environment, many methods of heat removal may not be reliable. For example, if the ambient air is hot, then using fans to blow air over the device will not work well. Additionally, a system using fans may have issues with water damage during a rainstorm.

A preferred solution is to transfer the generated heat into the unprocessed treatment flow. In typical drinking water treatment applications, the treatment flow will be sufficiently cold as to effectively remove heat from the UV-LED system if somehow the treatment flow can be brought into thermal contact with the UV-LED system. Traditional mercury-vapor bulb UV disinfection systems already use the treatment flow itself to cool the bulbs. This is accomplished by placing the mercury bulbs into cylindrical protective quartz tubes. These quartz tubes are then allowed to protrude through a section of treatment-flow pipe designated as the UV disinfection chamber. As water flows through this section of pipe it passes around these quartz tubes, cooling the mercury bulbs and further being exposed to the UV light emission from the mercury bulbs. The cylindrical profile of the mercury bulb plays a critical role in the practicality of this design.

UV-LED arrays are primarily planar, since construction of high-power UV-LED systems in a cylindrical footprint is inefficient. Furthermore, UV-LEDs must be kept cooler than mercury-vapor bulbs, meaning that heat-transfer through the protective quartz tubes will be insufficient to satisfy the cooling requirements of the UV-LED array. UV-LED arrays then require a new approach to transfer heat from the array to the treatment flow. The present invention provides various embodiments of devices and methods by which this is accomplished.

EP3461793 to Khan et al. (“Khan”) describes a water treatment application using planar sources external to a transparent reactor; However, the approach to positioning the UVC LED array is to generate a customized spatial flux distribution though use of a curved array or even a planar array to produce a dose distribution. The present invention acknowledges the higher flux zones created by the planar source, and instead uses a tailored flow velocity path and reactor length to optimize the dose distribution. Khan is silent on how to solve the problem of cooling or providing power to the planar array and associated power supplies, which are non-trivial when dealing with large scale arrays that require kilowatts of power.

U.S. Pat. No. 10,604,423 to McNulty describes cooling an array of LEDs positioned radially around a UV transmissive tubular reactor. However, this invention relies on focusing via collimating the UV radiation radially around the reactor to direct the radiation along the longitudinal axis toward the center of the pipe and concentrating the UV light there. This contrasts with the present invention, where the UV is not collimated, and the highest UV flux is generated on the outside of the inner diameter of the reactor which is directly opposite the UV array. This is then compensated by use of the flow mixing element that forces the fluid to pass into the regions of high UV flux, and the length of the tube ensures adequate mixing. Also uniquely described herein is inducing a rotational or vortical motion using a mixing element in the fluid upstream of the unit to ensure adequate treatment.

Notwithstanding, improvements in commercial fluid treatment using ultraviolet light are of continuing interest. A design of the reactor hydraulics should overcome deficiencies in fluence rate uniformity, not only in high-conversion reactions (e.g. disinfection which is measured as a log reduction) but also in mass transport limited operations (such as can be observed for highly absorbing fluids). It would likewise be beneficial to provide a resilient design that can maintain high efficiency of operation across a wide range fluid absorbances; this contrasts with applications where optimization of the fluence rate field uniformity is the principal route to highly efficient operation, since the fluence rate distribution is inherently connected and strongly dependent on the absorbance of the fluid under irradiation.

### SUMMARY OF THE INVENTION

The present invention provides a reactor utilizing the high-power density of UV-LEDs arranged into a planar array, to irradiate treatment fluid flowing through a cylindrical reaction (described hereinafter as the “irradiation chamber”) formed of a transmissive material. The planar array benefits significantly in simplifying production of the device, and the thermal control and power supply of the UV-LEDs.

The transmissive irradiation chamber may be enclosed within a secondary optical chamber of a reflective material, or coated or covered with a reflective material. Such a design maximizes optical in-coupling, by providing reflective surfaces to “redirect” radiation from the divergent planar source, which does not directly enter the irradiation chamber and the treatment flow, and secondarily acts to “recover” radiation that has passed into the irradiation chamber and exited through the transmissive wall. Such recovery of radiation into the irradiation chamber reduces net optical out-coupling, and therefore enhances operational efficiency. Materials providing reflective surfaces preferably have a bulk reflectivity of between 80% and 100% in the UV-C region emitted by the UV source.

The present invention provides fluid treatment apparatus and system for processing a treatment flow with ultraviolet (UV) light. The treatment of the treatment flow can include sanitizing the treatment flow.

The fluid treatment apparatus includes a cylindrical irradiation chamber consisting of an annular wall of a UV light-transmissive material. The annular wall has an outer surface, an inner surface, first and second opposing open ends forming a treatment flow path having a centerline.

The fluid treatment apparatus can include an inlet for the treatment flow into the first open end of the irradiation chamber, and an outlet out of the second open end of the irradiation chamber.

The fluid treatment apparatus also includes a planar array of UV-light emitting diodes (LEDs) configured to emit UV light. The planar array of UV-LEDs has a length along a centerline that is parallel to the centerline of the irradiation chamber, and a width. Preferably, a normal line of the planar array passes through both the centerline of the planar array and the centerline of the irradiation chamber.

The emitted UV light from the planar array of UV-LEDs impinges an arc segment of the outer surface along a length of the irradiation chamber, and passes through the annular wall and into the treatment flow path.

In some embodiments, the planar array of UV-LEDs has a width not greater than an outer diameter of the annular wall of the irradiation chamber.

In some embodiments, the planar array of UV-LEDs has an area of less than 60% of the area of the outer surface of the irradiation chamber.

The fluid treatment apparatus can include a light-reflecting material covering a non-irradiated portion of the outer surface of the irradiation chamber, not within the arc segment, and upon which UV light emitted directly from the planar array of UV-LEDs does not impinge. The light-reflecting material reflects UV light, which has passed from within the treatment flow path through the annular wall, back into and through the annular wall, returning the reflected UV light back into the treatment flow path.

In some embodiments, the light-reflecting material comprises one or more reflecting panels that are positioned against or over the non-irradiated portion of the outer surface. The one or more reflecting panels can be fixed directly onto the outer surface of the irradiation chamber or positioned in proximity thereto. In some embodiments, the light-reflecting material can be a reflective film or paint applied and adhered directly over the outer surface of the irradiation chamber. In some embodiments, non-irradiate portions of the outside surface of the quartz wall can be patterned or etched to create a sub-wavelength reflector that reflects UV light back into the irradiation chamber.

The fluid treatment apparatus can also include a heat exchanger apparatus configured to remove heat generated by the planar array of UV-LEDs. In some embodiments, one or more heat exchanger apparatuses are configured to remove heat generated by the planar array of UV-LEDs and, optionally though preferably, heat generated by one or more of a power supply unit and a control unit that power and control the operation of the planar array of UV-LEDs.

In some embodiments, the fluid treatment apparatus includes an LED array module that comprises the planar array of UV-LEDs and the heat exchanger apparatus. In some embodiments, the LED array module also includes a power supply for powering electrically the planar array of UV-LEDs, and optionally a control board for controlling the operation of the UV-LEDs.

In some embodiments, the fluid treatment apparatus includes a secondary chamber within which is enclosed the irradiation chamber. The secondary chamber includes an inlet opening and an outlet opening for access to the first open end and the second open end of the irradiation chamber.

The secondary chamber also includes a first opening for mounting the LED array module. In some embodiments, the opening is a rectangular flanged opening through which the planar array of UV-LEDs is exposed to the irradiation chamber.

In some embodiments, the fluid treatment apparatus can include a second planar array of UV-LEDs or more, and a corresponding second heat exchanger apparatus. In such embodiments, a secondary chamber can include a second flanged opening on an opposite lateral side of the first flanged opening.

In some embodiments, the second flanged opening are configured to orient the plane of the second planar array of UV-LEDs relative to the plane of the first planar array of UV-LEDs by an arc angle of 120-160 degrees.

In some embodiments, the heat exchanger apparatus comprises a cold plate having a first cold surface and an opposed second cold surface, wherein a planar array of heat sinks of the planar array of UV-LEDs are in thermal contact with the first cold surface, and the power supply is in thermal contact with the second cold surface.

In some embodiments, the fluid treatment apparatus further includes a means for swirling the untreated treatment flow at least one revolution through an emission portion of the length of the irradiation chamber.

In some embodiments, the fluid treatment apparatus includes a second planar array of UV-LEDs, and a corresponding second heat exchanger apparatus.

The apparatus, water treatment system, and a method of using the apparatus, is particularly suitable for drinking water treatment, wastewater treatment treatment of industrial process water and other applications requiring high fluid flow rates of fluid to be treated. In a preferred embodiment, each apparatus may be configured to treat 15 to 750 cubic meters of water per hour. Preferably, each apparatus may be configured to treat 30 to 500 cubic meters of water per hour. Most preferably, each apparatus may be configured to treat approximately 250 cubic meters of water per hour.

In addition to treating and sanitizing water and water supply systems, the apparatus of the present invention can be used for the industrial and commercial treatment of other liquids that are susceptible to contain or be contaminated by pathogens, including but limited to beverages, cleaning solutions, cooking products, and personal care products.

The invention also provides a water treatment system comprising a plurality of the fluid treatment apparatuses, arranged in parallel, in series, or in a combination thereof.

The present invention provides a fluid treatment apparatus for processing a treatment flow, comprising: a) a cylindrical irradiation chamber comprising a quartz wall of a UV light-transmissive material, having an outer surface, forming a treatment flow path; b) a planar array of UV-light emitting diodes (LEDs) placed to surround only a portion of the exterior of the quartz wall of the irradiation chamber, and configured to emit UV light that passes through the quartz wall; c) a UV-light reflective material covering the exterior of the quartz wall of the irradiation chamber not surrounded by the planar array of UV-LEDs, to reflect UV light that passes through the quartz wall back into the treatment flow; and d) a heat transfer system configured to removed heat energy from heat generating devices, including the planar array of UV-LEDs. In some embodiments, the planar array of UV-LED has a maximum width not exceeding an outer diameter of the annular wall of the irradiation chamber.

The nature and advantages of the present invention will be more fully appreciated from the following drawing, detailed descriptions, and claims.

### BRIEF DESCRIPTION OF THE DRAWINGS

The accompanying drawing illustrates embodiments of the invention and, together with a general description of the invention given above, and the detailed description given below, explain the principles of the invention.

FIG. 1 illustrates a fluid treatment apparatus of the present invention.

FIG. 2 illustrates an exploded view of the components of the fluid treatment apparatus of FIG. 1.

FIG. 3 illustrates a front elevation view of the fluid treatment apparatus.

FIG. 4 illustrates a sectional view of the fluid treatment apparatus transverse to the longitudinal centerline, taken along line 4-4 of FIG. 1.

FIG. 5 illustrates a sectional view of the fluid treatment apparatus along the longitudinal centerline, taken along line 4-4 of FIG. 1.

FIG. 6 illustrates the sectional view of FIG. 3 showing the positioning only of the irradiation chamber, the planar array of UV-LEDs, and the reflective panel and brackets, and a portion of the UV light radiation.

FIG. 7 illustrates an exploded outside view of an LED array module and its components.

FIG. 8 illustrates an exploded inside view of an LED array module and its components.

FIG. 9 illustrates a sectional view of the heat exchanger apparatus, taken along line 9-9 of FIG. 7.

FIG. 10 illustrates a sectional view of the heat exchanger apparatus, taken along line 10-10 of FIG. 7.

FIG. 11 illustrates an embodiment of a heat transfer system used with the fluid treatment apparatus for cooling the cold plate of the heat exchanger apparatus.

FIG. 12 illustrates another embodiment of a heat transfer system.

FIG. 13 illustrates another embodiment of a heat transfer system.

FIG. 14 illustrates another embodiment of a heat transfer system.

FIG. 14 illustrates another embodiment of a heat transfer system.

FIG. 16 illustrates the impact of LED position on performance for different array designs.

### DETAILED DESCRIPTION OF THE INVENTION

As used herein, the phrase “treatment flow” refers to fluid that is being exposed to UV light.

As used herein, the phrase “heat generating device” refers to any one, more, or all of UV-LEDs, electrical power sources, electronic control equipment, and any other heat-generating equipment associated with the UV-LED treatment apparatus and its operation.

The constraint of having the arrays of a large number of UV-LEDs in a largely planar arrangement creates unique design challenges, such as maintaining separation between the treatment flow and the UV-LED lamp, coupling of the emitted UV light into the treatment flow, ensuring that the treatment flow is uniformly exposed to the emitted UV light (explicitly not forming a uniform fluence rate field, rather balancing fluence rate non-uniformity with induced mixing, and maximizing optical coupling), and ensuring that the level of UV flux inside the chamber can be monitored.

The present inventive provides a fluid treatment apparatus that is designed to operate efficiently and adjust to the transmissivity of fluids flowing through the treatment chamber. For example, UV transmittance sensors across the flow cell from UV-LEDs can be added to the fluid treatment apparatus to relay information to a software system to turn off one or more individual, rows, or columns of LEDs 44 in high transmissivity fluids and turn them back on if the transmissivity decreases. For a system operating at constant, invariable UV output, it is necessary to always provide a UV output which will effectively treat the fluid at the highest possible temperature and lowest possible transmissivity to ensure that all of the fluid is effectively treated even in this worst case. However, because the energy usage of the system of the present invention can be limited by varying the UV output based on the temperature or transmissivity, the system can be operated at lower outputs when the temperature is lower than the maximum and/or transmissivity is higher than the minimum, thereby increasing operational efficiency.

It is to be understood that the present invention may be used in combination with oxidizing chemicals, such as chlorine, ozone, or hydrogen peroxide, injected into the fluid to carry out chemical and/or biological treatment.

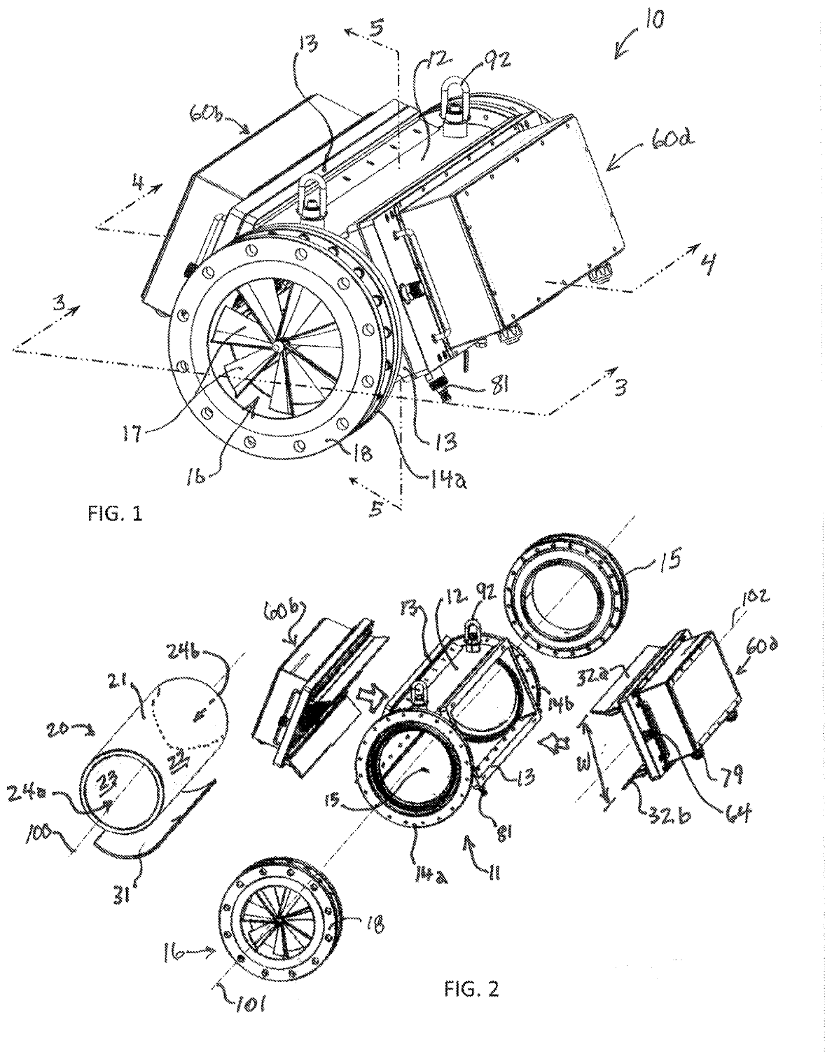

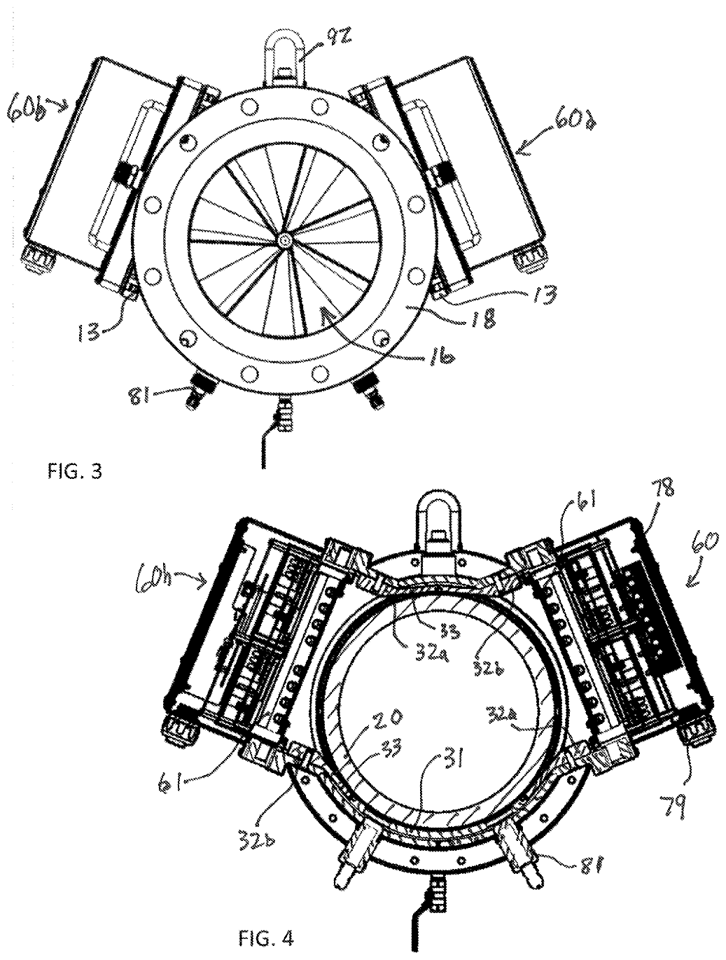

FIGS. 1-5 illustrate a fluid treatment apparatus 10 for processing a treatment flow with ultraviolet (UV) light.

### <Housing>

The apparatus 10 includes a main cylindrical housing 11 comprising a cylindrical wall 12 having a longitudinal centerline 101, a pair of opposed, flanged circular opening 14a and 14b on opposed ends of the cylindrical wall 12, and a pair of rectangular flanged openings 13 on the upper portions of the two opposite sides of apparatus 10 fixed along its rectangular periphery to the cylindrical wall 12. The cylindrical wall 12 also includes a pair of hoisting rings 92 fixed at the uppermost, opposites ends of the wall 12, a pair of UV sensors ports 15 on the two opposite sides of the lower portion of wall 12, and one or more drain openings in the bottom-most portion of the wall 12, along the vertical plane through the wall 12. In the illustrated embodiment, the UV sensors ports are positioned in the cylindrical wall 12 generally opposite the rectangular flanged openings 13.

### <Irradiation Chamber>

The cylindrical irradiation chamber 20 consists of an annular quartz wall that is transmissive to UV light. The quartz wall 21 has an outer surface 22, an inner surface 23, first and second opposing open ends 24a, 24b, providing a treatment flow path having a centerline 100. The thickness of the quartz wall is sufficient to withstand high treatment flow pressures, preferably up to about 1.0 MPascal, or more. The inner diameter of the quartz wall can be about 6 inches (15 cm), 8inches (20 cm) and 12 inches (30 cm), having wall thicknesses of from about 10 mm to about 20 mm, with a length of up to 120 cm, for example, 30-100 cm.

The coupling of the light from the planar array can be enhanced by choosing an appropriate size (longitudinal length and lateral width) of the planar array 40 relative to the radius of curvature of the outer diameter and inner diameter of the irradiation chamber 20. In some embodiments, the planar array of UV-LEDs has a width not greater than an outer diameter of the annular wall of the irradiation chamber.

In some embodiments, the planar array of UV-LEDs has an area of less than 60% of the area of the outer surface of the irradiation chamber; for example, 50% or less, or 40% or less, or 30% or less.

The quartz wall extends between the length of the opposed open ends of the quartz wall cylinder are positioned coaxially with the pair of opposed, flanged circular opening 14a and 14b, spaced with elastomeric o-rings, and sealed along its outer surface 23 to an inlet coupling flange 18 and an outlet coupling flange 15. Treatment flow supply and processed treatment flow discharge piping are sealingly fixed in fluid communication to the inlet coupling flange 18 and an outlet coupling flange 15, respectively, by gaskets or other well-known means, for example, with elastomeric o-rings and bolt securements. The gasket material can be a hybrid material of polytetrafluoroethylene (PTFE), the fluoroelastomer fluorine kautschuk material (FKM), or other suitable thermoset elastomer or thermoplastic, and stainless steel. The fluorinated materials comprising of carbon atoms surrounded by fluorine atoms, which gives them incredible chemical resistance.

### <Planar Array of UV-LEDs>

The fluid treatment apparatus also includes a pair of LED array modules 60a, 60b, each attachable removably to one of the pair of rectangular flanged openings 13. Each of the two LED array modules includes one or more conduit ports 79 to supply power and electronic control wiring, and a pair of flow ports 64 for separate supply and return piping of a cooling flow.

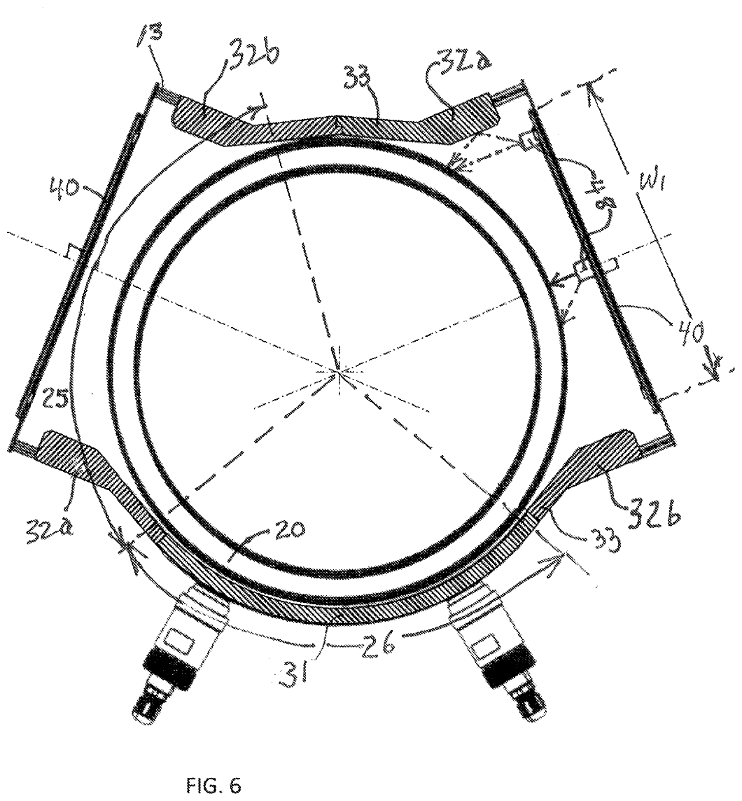

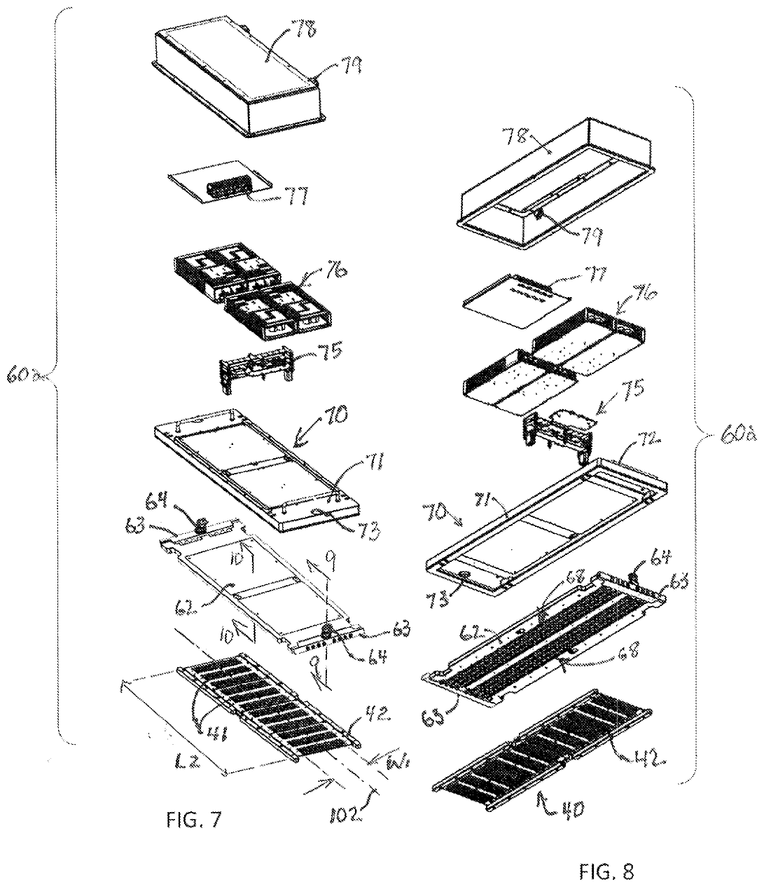

As shown in FIGS. 7 and 8, each LED array module 60 includes a planar array of UV-LEDs for emitting UV light to the interior of the cylindrical housing 11, and against the outer surface 23 of the quartz wall 21 of the irradiation chamber 20. The emitted UV light from the plurality of individual LEDs of the planar array impinges on the outer surface 23 of the quartz wall 21, which then passes through the quartz wall 21 and into the treatment flow path, as illustrated in FIG. 6.

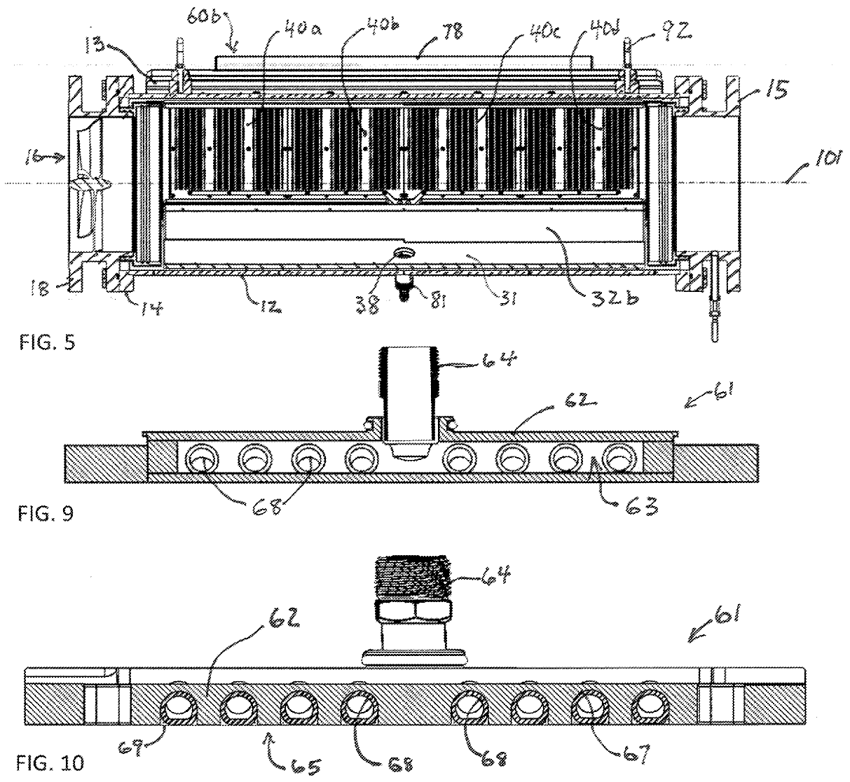

The planar array of UV-LEDs has a length L2 along a centerline 102 that is parallel to the centerline 100 of the irradiation chamber 20, and a width W2 (see FIG. 7). The planar array of UV-LEDs can include two or more small sub-arrays, arranged into an overall array pattern. Each planar array can include from one or more hundred to one or more thousands of individual LEDs 44. In a preferred embodiment, the planar array 40 includes four sub-arrays of UV-LEDs, each sub-array having from 1000 to 1500 individual LED lamps 44. Preferably, a normal line of the planar array 40 passes through both the centerline 102 of the planar array 40 and the centerline 100 of the irradiation chamber 20. The arrangement provides that a row of LEDs along the centerline 102 of the array emits UV light that is directed normally (line 103, FIG. 4) at the centerline 100 of the irradiation chamber 20, which impinges normally (perpendicularly) on the outer surface 22 of the quartz wall 21.

Positioning the planar array(s) 40 of UV-LEDs around a limited portion of the irradiation chamber 20 can make replacement of the planar UV emitters easier, in addition to making their manufacturing simpler.

The positioning of the LEDs is believed to be a key parameter for efficient coupling of the UV light source to the treatment flow, and therefore the spacing between rows of LEDs 44, known as LED pitch, can be optimized An LED pitch of greater than 1 mm between LED dies is preferable.

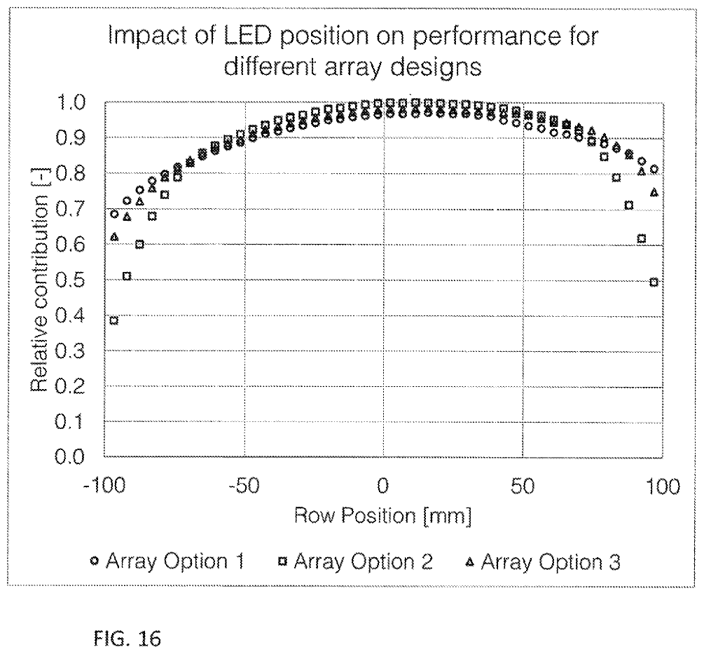

An exemplary case can be shown for the selection of LED position versus its contribution to the overall fluence of an irradiation chamber, illustrating the importance of source location (See FIG. 16). Here, the relative contribution of each LED is plotted for three array concepts as a function of row position (distance from LED die to the transparent irradiation chamber along a tangent to its surface); it can be seen the larger the displacement along the tangent (therefore greater misalignment of the LED normal to the radius of the irradiation chamber, for a planar array), the lesser the relative contribution of that LED to the overall performance. It can also be seen that elements of the array design may affect the gradient of this decline with increasing displacement, but also the peak of the contribution from centrally positioned LEDs in each design. This illustrates the art of the co-design of the planar array and irradiation chamber required to maximize efficiency and/or efficacy. Variation of the planar array of UV-LEDs may optionally include design of reflective elements surrounding the periphery of the planar array which preferentially redirect the emitted UV radiation towards the treatment flow, as embodied in the reflective brackets described herein. The planar array options of FIG. 16 illustrate the impacts of some of these features, such as the PTFE reflector brackets 32 as shown in FIG. 4. The emissions of LEDs displaced far along the tangent (towards the lateral edges of the planar array 40) can be reflected and recycled into the treatment flow, as shown in FIG. 6, whereas without the would have been absorbed by other elements and wasted.

### <Reflective Coverings>

The outer non-irradiated surface(s) of the quartz wall 21 that are not directly exposed to the emitted UV light can be instead surrounded, covered, or coated by a UV reflective material, which improves reactor performance by reflecting UV light that passes through the quartz wall back into the treatment flow, and providing a more homogeneous distribution of the UV flux inside the irradiation chamber. Non-limiting examples of the UV reflective material include polytetrafluoroethylene (PTFE), aluminum, a metallized thermoplastic material, or any other can be any highly reflective material preferably have a bulk reflectivity of between 80% and 100% in the UV-C region emitted by the UV source.

One or more light-reflecting materials are provided to cover a portion or portions of the outer surface 22 of the irradiation chamber 20. Portions on the which the UV light emitted directly from the UV-LEDs are not covered by the light-reflecting materials. The light-reflecting materials cover only and preferably all the remaining outside surfaces 22, to reflect, back into the irradiation chamber 20, the refracted and diffused UV light that passes from the irradiation chamber out through the quartz wall 21. In the illustrated embodiment, the light-reflecting material comprises one or more reflective panels that are positioned against or over the non-irradiated portion of the outer surface. In some embodiments, the reflective panels are displaced or spaced away from the outer surface 22 of the quartz wall 21 by an airspace.

A first reflecting panel provides a guide or shield that restricts the impingement of the emitted UV light to an area of the outer surface of the quartz wall. In the illustrated embodiment, for each planar array 40, a pair of reflector brackets 32a, 32b are fixed inside the cylindrical housing 11 along the upper and lower edges of the flanged openings 13. A blade portion 33 of each reflector bracket 32a, 32b extends to engage tangentially the outer surface 22 of the quartz wall 21 (FIGS. 4 and 6) to restrict the impingement of the UV light to an uncovered area consisting of an arc segment 25 along the length of the quartz wall 21, which faces the planar array 40 of UV-LEDs. During treatment flow processing, the reflective side surfaces of the reflector brackets 32 reflect any angled emitted light from the UV-LEDs, particularly those at the periphery of the planar array 40, towards the uncovered area.

In some embodiments, the planar arrays of UV-LEDs only surround a portion of the irradiation chamber 20, and only emits UV light directly only onto a portion of the peripheral outer surface of the quartz wall 21. During treatment flow processing, a portion of the UV light that has entered the treatment flow path within the irradiation chamber 20 will be diffused and/or refracted outwardly and penetrate the inner surface 23 of, and through, the quartz wall 21. Another reflector panel 31 is placed over the outer surface 22 of the quartz wall 21 in any remaining areas where the emitted UV light is not directly impinging the quartz wall. In the illustrated embodiment, a reflector panel 31, as shown in FIGS. 1 and 4, covers the underside surface of the quartz wall.

In some embodiments, the lateral confronting edges of the adjacent reflector panel 31 and/or reflector brackets 32 are configured to overlap one another, to prevent UV light from escaping therebetween.

In some embodiments, an opening 37 is made in a reflector panel 31 to provide an optical access to the outer surface of the quartz wall, for a UV light sensor 81, as shown in FIG. 4. The opening 37 enables placement of the UV light sensor directly against the outer surface 22 of the quartz wall 21.

### <LED Array Modules>

In some embodiments, the LED array module also includes and houses one or more other systems for providing power, control and/or cooling of the UV LEDs of the planar array. Arranging the planar array 40 of LEDs and these other systems in a unitary module allows for quickly replacing spent or defective LEDs or power supply components during operation or scheduled maintenance.

In the illustration shown in FIGS. 7 and 8, the LED array module 60 (in this case, the first module 60a) houses and interfaces, in stacked order, the planar array 40 of UV-LEDs, a heat exchanger apparatus 61, a control unit 75, a power supply unit 76 for powering electrically the planar array 40 of UV-LEDs, a power bus 77, and a module cover 78.

The planar array of UV-LEDs (also described here as a planar UV emitter) is typically in an array of UV-emitting LEDs fixed to printed circuit boards (PCBs) 41 and supported on frame 42, to provide a planar heat sink for drawing heat away from the LEDs.

### <Heat Exchanger Apparatus>

The fluid treatment apparatus includes a heat exchanger apparatus configured to remove heat generated by the planar array of UV-LEDs. In some embodiments, a heat exchanger apparatus, or a second heat exchanger apparatus, is configured to remove heat generated by planar UV emitters, and optionally though preferably by the one or more power supply units and control units that power and control the operation of the planar array of UV-LEDs.

The heat exchanger apparatus 61 comprises a cold plate having a first cold surface and an opposed second cold surface, wherein the planar heat sinks of the planar array 40 of UV-LEDs are in thermal contact with a first planar cold surface 65 of a cold plate 62. A thermal adhesive 66 provides a thermal conductive interface between the planar heat sinks and the first cold surface 65. Thermal contact is maintained by securing the planar arrays 40 to the first cold surface of the heat exchanger apparatus using removable fasteners, such as bolts.

In preferred embodiments, the cold plate is made of aluminum or other highly thermally-conductive metal.

The cooling of the cold plate 62 is provided by a plurality of elongated passages through the length of the cold plate. In some embodiments, the passages consist of elongated tubes 68 laid into a corresponding plurality of parallel slots 67 formed longitudinally into the first cold surface 65, as shown in the sectional view of the cold plate of FIG. 9. A pair of opposed manifolds 63 is positioned on opposite ends of the cold plate, each manifold in sealed fluid communication with the one end of the plurality of tubes 68 through a joint tube, as shown in the sectional view of the cold plate of FIG. 10. Cooling water is supplied to, and heated water is removed from, the opposed manifolds 63 through respective flow ports 64. The flow ports 64 are attached in fluid communication to a heat exchanger system to deliver cooling water, and to remove heated water, described herein elsewhere.

The elongated tubes are copper tubing, with copper being a more acceptable water-contact material than aluminum. In another embodiment, or the elongated passages can be formed by milled directly into the cold plate.

The upper cooling surface of the cold plate 62 is covered in direct contact with a cooling cover 70. Both the planar arrays 40 and the heat exchanger apparatus are secured by fasteners, such as bolts, to the inner side of the frame 71 of the cooling cover 70. The outer surface of the cooling cover 70 provides cooling to the power supply 76 and the control unit 75 that are fixed directly and secured by fasteners. A power bus 77 is electrically connected and physically attached to the power supply 76. Finally, a component cover 78 covers the control unit 75, the power supply 76 and the power bus 77, and is fixed to the upper surface of the cooling cover 70 using fasteners such as bolts. An electrical conduit 79 secured through the cover 78 provides access for power and control to the module.

In a preferred embodiment, the components on each side of cold plate 62 are individually maintainable and/or removable to reduce the quantity of consumable elements during and maintenance or retrofitting operations.

Using the cold plate as both a structural element to support the planar array of UV-LED and the power supply and controller components is optimal. The components of the apparatus 10 most likely to fail (the UV-LEDs and the electronics) are more easily replaced by simply disconnecting the power and controller wiring and removing the LED array module 60. Furthermore, coupling the power supply and electronic controller elements to the heat exchanger apparatus 61 allows their thermal load to be managed without adding additional cooling elements. The electronics can be largely sealed within a cover 78 increasing protection and resistance to environmental risks. Installation is made simpler because only power supply and control signals need to be run to the LED array module 60. In conventional UV products the power and control electronics are installed within a separate shielded enclosure. Placing all fragile elements (electronics, UV-LED arrays) into a single component (the LED array module) simplifies production.

### <In-Flow Swirling>

To further improve the distribution of the UV flux to the treatment flow inside the irradiation chamber, a swirling motion can be imparted into the treatment flow before it enters the quartz tube, such that the impact of high-UV intensity areas directly in front of the planar UV emitters is minimized. This swirling motion can be generated by a static flow-mixing element, such as a vortex generator, situated in the treatment flow, upstream of the quartz tube.

One embodiment of a flow mixing element can be a vaned impeller that creates a vortex in the flow, coupled to the inlet of the irradiation chamber. The shape and count of the vanes can be modified to create the required vortex down the length of the irradiation chamber, to ensure that the fluid volume achieves a uniform dose of UV radiation. In vacations embodiments, the size and positioning of the flow mixing element can be selected according to the treatment flow rate and/or the inlet diameter of the irradiation chamber.

The deployment of a cylindrical irradiation chamber allows for the deployment of flow conditioning elements such as the vortex generators, since the chamber has a consistent cross section; vortex generators introduce a swirling laminar flow region which causes the flow to spiral along the length of the irradiation chamber. In some embodiments, the vortex generator comprises between 4 and 10 vanes that result in at least I rotation of the fluid along the length of the irradiation chamber under nominal operating conditions. In this embodiment, since a discrete number of planar UV-LED arrays are employed, which do not cover the entire surface the transmissive irradiation chamber, a highly non-uniform fluence rate field is produced with ‘hot spots’ adjacent to the array locations and “dark regions” at other places. The dose to the fluid is achieved via an integration of the fluence between the hot spots of high UV flux and dark regions of low UV flux. By optimizing the pitch of the vortex generator, the swirling laminar flow may be tuned to complete a desired number of rotations along the length of the irradiation chamber, and in doing so “integrate” exposure equally between these hot spots and dark regions. The resulting design has both high optical efficiency through strong in-coupling and weak out-coupling, and high hydraulic efficiency, leading to an overall high reaction efficiency, high operating efficiency, and low operating power consumption.

### <UV Sensor>

The present invention provides a method for measuring UVT using dual sensor positions and cycling output between the dual sensors, using the known relationship between lamp output and sensor. Large-scale UV fluid treatment for both photochemical, sanitization and disinfection purposes requires a very high flux of UV light. Conventional mercury vapor lamp systems typically utilize a cylindrical lamp. These lamps can be placed directly into the treatment flow path, and arranged to achieve a consistent distribution of UV light within the flow path. This light distribution ensures a predictable level of disinfection within the treatment flow.

In some embodiments, the percentage of light that the UV sensor can report on can be lowered.

### <Heat Transfer Systems>

The present invention further provides a means for using a heat transfer system to balance the specifications/requirements of the treatment flow with the specifications/requirements of the heat generating devices, while optimizing for cost/simplicity. Treatment flow specifications can include temperature, chemistry, and suspended solid; heat generating specifications can include heat power output; and heat transfer system requirements can include steady-state operating temperature and ease of maintenance.

The heat transfer system can be integrated with the fluid treatment apparatus 10 to provide a supply of cooling flow to the heat exchanger apparatus 40. Typically the volumetric flow rate of the cooling flow (3-20 gallons per minute, gpm) is very small in comparison to the treatment flow. The heat transfer system can include a cooling flow metering device that ensures the cooling flow rate is sufficient for cooling, but not excessive/wasteful. In one embodiment, a metering valve can reduce the gauge pressure of the cooling flow within the cold plate, such that the pressure of the cooling flow is lower than the pressure of the treatment flow.

A heat transfer system described herein can include one or more temperature sensors for monitoring and/or controlling of the temperature of the cooling flow.

In some embodiments when the treatment flow is used as the cooling flow, the cooling flow is taken from the processed treatment flow down-stream of the fluid treatment apparatus 10, such that the cooling flow passing through the cold plate of the heat exchanger apparatus has been UV-treated, which decreases the opportunity for fouling within the cold plate channels. In other embodiments, the cooling flow can be taken from the untreated treatment flow up-stream of the fluid treatment apparatus 10. Embodiments illustrated herein showing the cooling flow from either upstream or downstream of the fluid treatment apparatus 10 would apply equally as well to the other.

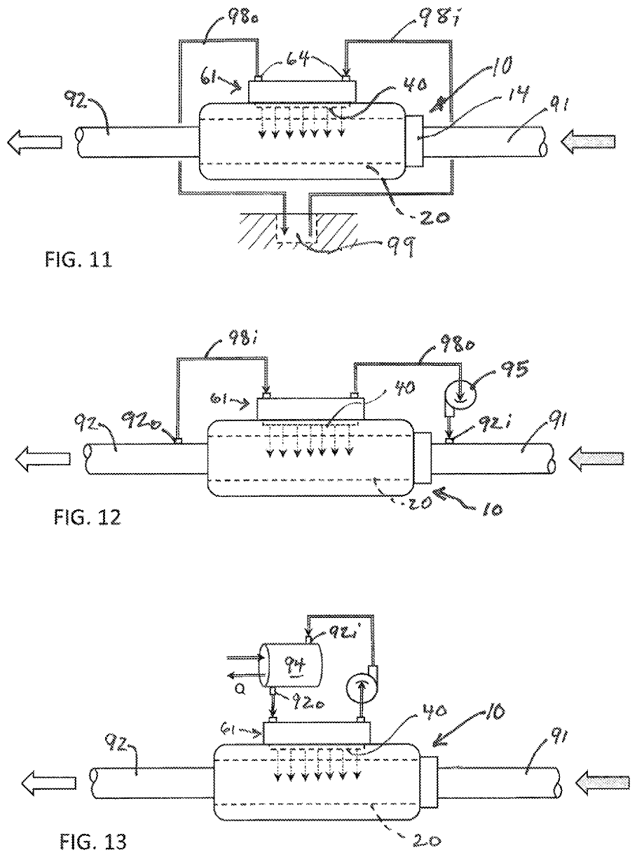

In a first embodiment shown in FIG. 11, a small pipe 98i is connected between a supply piping 91 carrying the treatment flow, and an inlet 64 of the heat exchanger apparatus 40. An outlet 64 of the heat exchanger apparatus 40 is channeled to a reservoir 99. This reservoir 99 can be an underground well, for example, from which the treatment flow was originally sources and pumped. The difference between the high pressure of the treatment flow at the supply piping 91 and the lower pressure of the reservoir 99 causes a flow of cooling flow (cooling water) through the cold plate 62 of the heat exchanger apparatus 61, cooling the cold plate 62, and extracting heat from the heat sinks of the UV-LEDs of the planar array 40, and optionally from the power supply 76 and controller electronics 75. This system would require minimal changes to existing customer installations.

For practical purposes, the reservoir 99 must be at a lower gauge pressure than the treatment flow to induce cooling flow through the heat exchanger apparatus 61. After the cooling flow has passed through the cold plate 61 and absorbed heat energy, it must be discharged. An optimal choice for a fluid treatment apparatus 10 installed directly on the outlet of a municipal well would be to dump the cooling flow outlet from the heat exchanger apparatus 61 back into the well. In this case, the well would be the reservoir. In intermittent usage scenarios, the cooling flow could simply be dumped onto the ground or into a small ditch.

A second embodiment shown in FIG. 12 is similar to the embodiment described immediately above, except that a suitable supply reservoir is neither available nor needed. A small portion of the treatment flow from downstream of the fluid treatment apparatus 10 is passed through the heat exchanger apparatus 40 before being re-injected into the treatment flow stream at a point upstream of the apparatus 10. Because there is a small and perhaps negligible pressure difference between cooling flow supply and return, a pump 95 is used to create a pressure differential in the cooling flow between the inlet and the outlets of the heat exchanger apparatus 40.

Alternatively, a cooling flow pressure differential can be created using a cooling flow supply piping (treatment flow takeoff) shaped as a scoop, facing into the treatment flow direction, or a venturi or pitot tube at a cooling flow return (treatment flow return). The pitot or venturi effect can provide a sufficient pressure differential to suction a small though sufficient portion of the treatment flow into the cooling flow inlet.

In some applications there may be limitations which prevent allowing the treatment flow from coming into direct contact with the cold plate. For example, the cold plate 61 can be aluminum, and water-contact material regulations may disallow treatment flow contact with aluminum. The treatment flow chemistry can also prove corrosive to the cold plate materials. Solids within the treatment flow can pose a risk of clogging or damaging the cold plate.

FIG. 13 illustrate a secondary closed-loop heat exchange subsystem that exchanges heat from the heat exchanger apparatus 61 with a separate, secondary heat exchanger system 94. The cooling flow within the secondary heat exchanger system is circulated using a pump 95 that circulates cooling flow from the outlet port 64 of the heat exchanger apparatus 61 into an inlet 92i of the secondary heat exchanger system 94, where the heat acquired from the planar array 40 of the UV-LEDs is removed. The cooling flow exits an outlet port 920 of the secondary heat exchanger system 94 and passes into the inlet port 64 of the heat exchanger apparatus 61. These cooling systems avoid contact or mixing between the closed loop cooling flow and the treatment flow, and thus can be a fluid specifically engineered to decrease freezing risks, decrease corrosion, etc., thereby increasing the design freedom of the cold plate 62. In some embodiments, the cooling flow liquid can comprise a mixture of glycol and water. The pump 95 can be driven by an electric motor, a water wheel or turbine powered by the treatment flow, a hydraulic drive, or a compressed air drive. Preferably the pump is placed between the outlet of the heat exchanger apparatus 61 and the cooling flow return to the treatment flow to reduce gauge pressure within the cold plate.

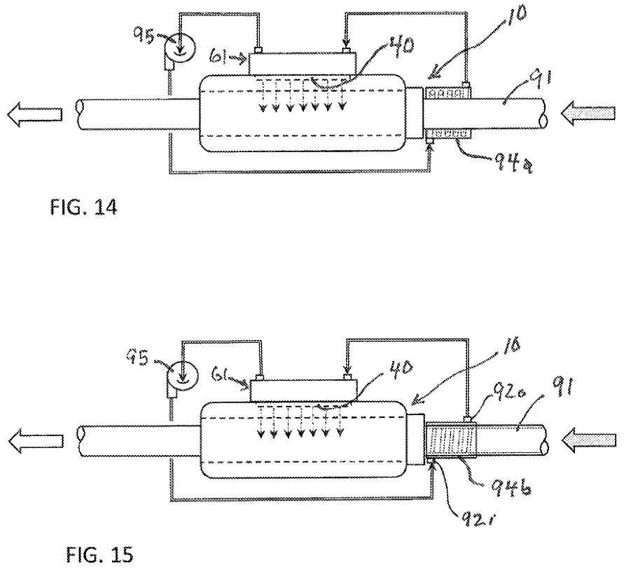

In another embodiment shown in FIG. 14, the secondary heat exchanger 94a can comprise a section of a stainless-steel pipe, through which the treatment flow passes, with separate cooling-flow tubing wrapped around the exterior surface of the supply piping 91 (such as copper tubing) through which the cooling flow passes. Heat from the cooling flow passes into the stainless-steel pipe and subsequently into the treatment flow. To enhance the efficiency of the secondary heat exchanger 94a, the inner surface of the stainless pipe can be structured to increase the amount of surface area in contact with the treatment flow, such as with machining fins or trenches.

In another embodiment shown in FIG. 15, the secondary heat exchange 94b can be a section of a stainless-steel pipe, having an inlet manifold in communication with the inlet port 92i, an outlet manifold in communication with the outlet port 92o, and a plurality of exchange tubing in flow communication between the inlet manifold and the outlet manifold, and the outside surfaces of the exchange tubing in fluid contact with the treatment flow.

While the present invention has been illustrated by the description of embodiment examples thereof, it is not intended to restrict or in any way limit the scope of the appended claims to such detail. Additional advantages and modifications will be readily apparent to those skilled in the art. Accordingly, departures may be made from such details without departing from the scope of the invention.

1. A fluid treatment apparatus for processing a treatment flow, comprising:
a) a cylindrical irradiation chamber comprising a quartz wall of a UV light-transmissive material, having an outer surface, forming a treatment flow path;
b) a planar array of UV-light emitting diodes (LEDs) placed to surround only a portion of the exterior of the quartz wall of the irradiation chamber, and configured to emit UV light that passes through the quartz wall;
c) a UV-light reflective material covering the exterior of the quartz wall of the irradiation chamber not surrounded by the planar array of UV-LEDs, to reflect UV light that passes through the quartz wall back into the treatment flow; and
d) a heat transfer system configured to removed heat energy from heat generating devices, including the planar array of UV-LEDs.

2. The fluid treatment apparatus according to claim 1, wherein the planar array of UV-LED has a maximum width not exceeding an outer diameter of the annular wall of the irradiation chamber.

3. A fluid treatment apparatus for processing a treatment flow, comprising:
a) a cylindrical irradiation chamber comprising an annular wall of a UV light-transmissive material, having an outer surface, an inner surface, first and second open ends forming a treatment flow path, and a centerline;
b) a planar array of UV-light emitting diodes (LEDs) configured to emit UV light, having a length with a centerline parallel to the centerline of the irradiation chamber, and a width, and wherein the emitted UV light impinges and passes through an arc segment of the outer surface of the length of the irradiation chamber, through the annular wall, and into the treatment flow path;
c) a light-reflecting material covering a non-irradiated portion of the outer surface of the irradiation chamber not within the arc segment, to reflect UV light that passes from the treatment flow path through the annular wall back into and through the annular wall; and
d) a heat exchanger apparatus configured to removed heat generated by the planar array of UV-LEDs.

4. The fluid treatment apparatus according to claim 3, wherein the width of the planar array of LED-UV emitters is not greater than an outer diameter of the annular wall of the irradiation chamber.

5. The fluid treatment apparatus according to claim 1, wherein the planar array of UV-LEDs has an area of less than 60% of the area of the outer surface of the irradiation chamber.

6. The fluid treatment apparatus according to claim 1, wherein a normal line of the planar array passes through both the centerline of the planar array and the centerline of the irradiation chamber.

7. The fluid treatment apparatus according to claim 1, further including an LED array module comprising the planar array of UV-LEDs, the heat exchanger apparatus, a power supply for powering electrically the planar array of UV-LEDs, and a control board for controlling the operation of the UV-LEDs.

8. The fluid treatment apparatus according to claim 1, wherein the irradiation chamber is enclosed within a secondary chamber, the secondary chamber including an inlet opening and an outlet opening for access to the first open end and the second open end of the irradiation chamber, and a first flanged opening for mounting the LED array module.

9. The fluid treatment apparatus according to claim 1, further including a second planar array of UV-LEDs, and a corresponding second heat exchanger apparatus.

10. The fluid treatment apparatus according to claim 1, wherein the secondary chamber includes a second flanged opening on an opposite lateral side of the first flanged opening, configured to orient the plane of the second planar array of UV-LEDs relative to the plane of the first planar array of UV-LEDs by an arc angle of 120-160 degrees.

11. The fluid treatment apparatus according to claim 1, wherein the heat exchanger apparatus comprises a cold plate having a first cold surface and an opposed second cold surface, wherein a planar array of heat sinks of the planar array of UV-LEDs are in thermal contact with the first cold surface, and the power supply is in thermal contact with the second cold surface.

12. The fluid treatment apparatus according to claim 1, further including a means for swirling the untreated treatment flow at least one revolution through the emission portion of the length of the irradiation chamber.

13. The fluid treatment apparatus according to claim 3, wherein the planar array of UV-LEDs has an area of less than 60% of the area of the outer surface of the irradiation chamber.

14. The fluid treatment apparatus according to claim 3, wherein a normal line of the planar array passes through both the centerline of the planar array and the centerline of the irradiation chamber.

15. The fluid treatment apparatus according to claim 3, further including an LED array module comprising the planar array of UV-LEDs, the heat exchanger apparatus, a power supply for powering electrically the planar array of UV-LEDs, and a control board for controlling the operation of the UV-LEDs.

16. The fluid treatment apparatus according to claim 3, wherein the irradiation chamber is enclosed within a secondary chamber, the secondary chamber including an inlet opening and an outlet opening for access to the first open end and the second open end of the irradiation chamber, and a first flanged opening for mounting the LED array module.

17. The fluid treatment apparatus according to claim 3, further including a second planar array of UV-LEDs, and a corresponding second heat exchanger apparatus.

18. The fluid treatment apparatus according to claim 3, wherein the secondary chamber includes a second flanged opening on an opposite lateral side of the first flanged opening, configured to orient the plane of the second planar array of UV-LEDs relative to the plane of the first planar array of UV-LEDs by an arc angle of 120-160 degrees.

19. The fluid treatment apparatus according to claim 3, wherein the heat exchanger apparatus comprises a cold plate having a first cold surface and an opposed second cold surface, wherein a planar array of heat sinks of the planar array of UV-LEDs are in thermal contact with the first cold surface, and the power supply is in thermal contact with the second cold surface.

20. The fluid treatment apparatus according to claim 3, further including a means for swirling the untreated treatment flow at least one revolution through the emission portion of the length of the irradiation chamber.
